(19) United States
(12) Patent Application Publication
(10) Pub. No.: US 2016/0287135 A1
(43) Pub. Date: Oct. 6, 2016

(54) BIOPSY NEEDLE WITH SENSING ELECTRODE ARRAY AND METHOD FOR MANUFACTURING THE SAME

(71) Applicants: KOREA ADVANCED INSTITUTE OF SCIENCE AND TECHNOLOGY, Daejeon (KR); THE ASAN FOUNDATION, Seoul (KR)

(72) Inventors: In Kyu Park, Daejeon (KR); Jae Ho Park, Daejeon (KR); Sang Hyeok Kim, Daejeon (KR); Joon Beom Seo, Seoul (KR); Nam Kug Kim, Seoul (KR); Jae Soon Choi, Seoul (KR)

(21) Appl. No.: 14/677,028

(22) Filed: Apr. 2, 2015

(51) Int. Cl. A61B 5/06; A61B 5/00; A61B 5/053

(52) U.S. Cl. CPC A61B 5/068; A61B 5/0538; A61B 5/6848; A61B 2010/045

(57) ABSTRACT

Provided is a biopsy needle with an electrode array which measures impedance for a plurality of biopsy points in real time, in which the needle includes electrode patterns configured by a plurality of electrode arrays on the surface, and the electrode patterns are spaced apart from each other along the top and bottom of the needle. According to the present invention, it is possible to selectively measure a predetermined tissue around the needle according to a direction of electrode formation and the number of electrodes while measuring impedance by using a plurality of electrodes by proposing a biopsy needle with a plurality of electrode arrays on a surface.

### BACKGROUND OF THE INVENTION

1. Field of the Invention

The present invention relates to a biopsy needle capable of measuring an impedance of a biopsy tissue, and more particularly, to a biopsy needle with a sensing electrode array and a method for manufacturing the same capable of selectively measuring a predetermined tissue around the needle while measuring an impedance by using a plurality of electrodes.

2. Description of the Related Art

Currently, checking a position of an end of a biopsy needle during a biopsy largely depends on imaging equipment and experience of doctors. Meanwhile, there is also a part which may not be checked by only the imaging equipment, such as an inhomogeneous part in the same organ or a prostate cancer. Due to the reason, the accuracy of the biopsy tissue deteriorates, and a necrotic tissue and other inflammation tissues rather than a substantial tumor tissue are taken, and as a result, an additional tissue biopsy is clinically required in some cases.

Further, since a tissue of the local portion having relatively low malignancy in the same tumor tissue is taken, there is a problem in that it is determined that the malignancy of the tumor is low. (M. Gerlinger et al., 2012, New. Engl. J. Med., 366, 10, 628-634)

Accordingly, in order to increase a success rate of the tissue biopsy, techniques of analyzing a tissue distribution or characteristic around the biopsy needle in real time are required. International case studies of measuring electrical impedance of the tissue around the end of the biopsy needle have been reported (V. Mishra et al., 2013, The Prostate, 73, 1603-1613). In the case studies, since the biopsy needle is just used as the its own electrode, there is a limitation that only the two-electrode type measurement is possible, and there is a problem in that the reduction of measurement accuracy due to the electrode polarization phenomenon when the biopsy specimen is measured may not be solved.

In detail, the biopsy needle for measuring impedance used in the study of V. Mishra is electrically insulated except for only internal and external ends of the needle to analyze the tissue existing between the internal and external ends of the biopsy needle. In the biopsy needle, since the impedance of the entire tissue around the end of the needle is measured, it is difficult to selectively measure the local tissue at a predetermined position around the needle, and further, since only the two-electrode measurement is possible, the electrode polarization phenomenon occurs, and as a result, there is a problem in that the error of the impedance measurement is caused.

The contents may be verified in measured data of saline shown in papers of research teams other than V. Mishra. In detail, in the measurement data, a conductance value measured at a low frequency in the salt water having conductance of 0.4 (S/m) is gradually reduced and as a result, it can be seen that the measurement error due to the electrode polarization phenomenon occurs. Further, the local tissue around the needle may not be selectively measured, only an average electrical characteristic around the needle may be measured, and as a result, there is a problem in that the distribution of the impedance of the tissue around the needle may not be locally or selectively verified.

### SUMMARY OF THE INVENTION

The present invention has been made in an effort to provide a biopsy needle with a sensing electrode array and a method for manufacturing the same capable of selectively measuring a predetermined tissue around the needle according to a direction of electrode formation and the number of electrodes while measuring an impedance by using a plurality of electrodes by proposing a biopsy needle with a plurality of electrode arrays on a surface.

An exemplary embodiment of the present invention provides a biopsy needle with an electrode array which measures impedance for a plurality of biopsy points in real time, in which the needle includes electrode patterns configured by a plurality of electrode arrays on the surface, and the electrode patterns are spaced apart from each other along the top and bottom of the needle.

The plurality of electrode arrays may be patterned at different positions on the needle.

The electrode pattern may be a metal paste or a metal wire for the electrode including any one of gold, silver, and stainless steel.

A material of the needle may include stainless steel.

The electrode pattern may be a four-electrode type with four electrodes.

Electrical insulating layers may be formed between the electrode pattern and the needle and on the surface of the needle except for the electrode pattern portions.

Another exemplary embodiment of the present invention provides a method of manufacturing a biopsy needle with an electrode array, which measures impedance for a plurality of biopsy points in real time, the method including: preparing a needle body; and patterning electrode patterns configured by a plurality of electrode arrays on the needle body, in which the electrode patterns are spaced apart from each other along the top and bottom of the needle.

The patterning of the electrode patterns may include patterning a first electrode pattern on the needle body with a metal paste and depositing a second electrode pattern on the first electrode pattern by a plating method.

The first electrode pattern may be a silver paste, and the second electrode pattern may be a gold-plated pattern.

The method may further include forming an electrical insulating layer on the surface of the needle body except for the electrode pattern portions before and after the patterning of the electrode patterns.

The electrode pattern may be a four-electrode type with four electrodes.

Yet another exemplary embodiment of the present invention provides a method of measuring impedance of a biopsy tissue, including measuring a change in impedance which is measured in each electrode array through a process of sequentially inserting a plurality of electrode arrays forming the electrode patterns into the biopsy tissue.

According to the exemplary embodiment of the present invention, it is possible to selectively measure a predetermined tissue around the needle according to a direction of electrode formation and the number of electrodes while measuring an impedance by using a plurality of electrodes by proposing a biopsy needle with a plurality of electrode arrays on a surface.

Further, the electrodes are formed on the biopsy needle through screen printing and may be used as an impedance sensor for the biopsy tissue which is a peripheral material by using a pair of electrodes patterned on the needle.

Further, an impedance of the tissue around the needle during the biopsy is measured and analyzed in real time by using the needle capable of measuring the impedance to improve the accuracy of tissue sampling during a surgical procedure.

In the present invention, when the impedance of the peripheral tissue which contacts the biopsy needle is measured based on the fact that there is a difference in electrical impedance between a normal tissue and a cancer tissue, the tissue around the biopsy needle may be accurately determined. That is, an analysis of the tissue around the needle through the impedance is performed in real time during the biopsy to solve inaccuracy having an existing tissue biopsy.

The present invention overcomes disadvantages that it is difficult to selectively measure a local tissue at a predetermined position around the needle because the impedance of the entire tissue around the needle end is measured by a biopsy measuring method using an electrode needle in the related art through a four probe measurement method and an electrode polarization phenomenon occurs because only two-electrode measurement is performed to cause an error of the impedance measurement.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a schematic diagram of a biopsy needle according to an exemplary embodiment of the present invention.

FIG. 2 is a manufacturing process diagram for the biopsy needle of FIG. 1.

FIG. 3 is a photograph illustrating an actual product for the biopsy needle of FIG.

FIGS. 4A and 4B are diagrams illustrating an example of a biopsy needle for measuring impedance based on two electrodes and four electrodes and a result of measuring conductance of salt water.

FIGS. 5A and 5B are diagrams illustrating an experiment for measuring impedance of a local tissue using a biopsy needle with a multiple electrode arrays and an experimental result.

FIG. 6 is a schematic diagram of a biopsy needle according to another exemplary embodiment of the present invention.

FIG. 7 is a manufacturing process diagram of a needle using a stainless steel wire or a metal wire as an electrode material.

FIG. 8 is a schematic diagram for a method of arranging metal wires.

FIG. 9 is a photograph illustrating an actual example of arrangement of metal wires.

FIG. 10 is a process diagram in which arranged metal wires are attached onto the needle.

FIG. 11 is a photograph illustrating an actual product for the needle to which the metal wires are attached by using a transfer process and an adhesive.

FIG. 12 is a graph illustrating a result of measuring conductance of salt water by using the needle to which the metal wire electrode is attached.

In the following description, the same or similar elements are labeled with the same and similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

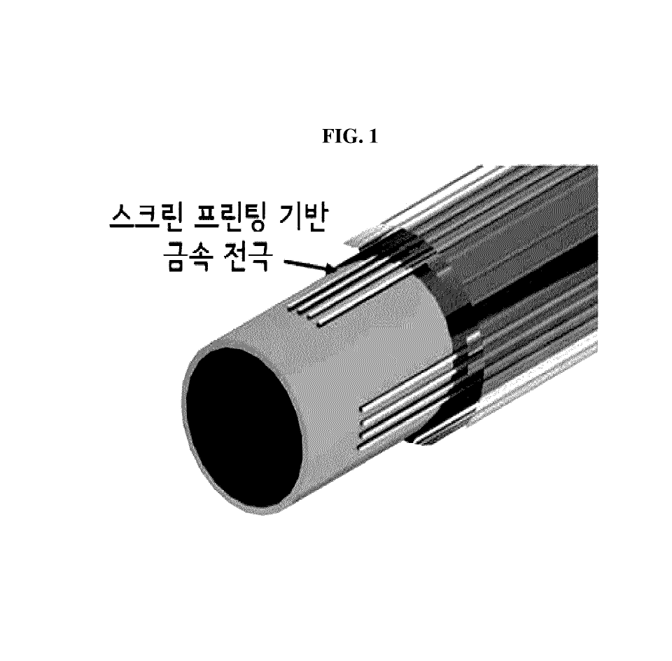

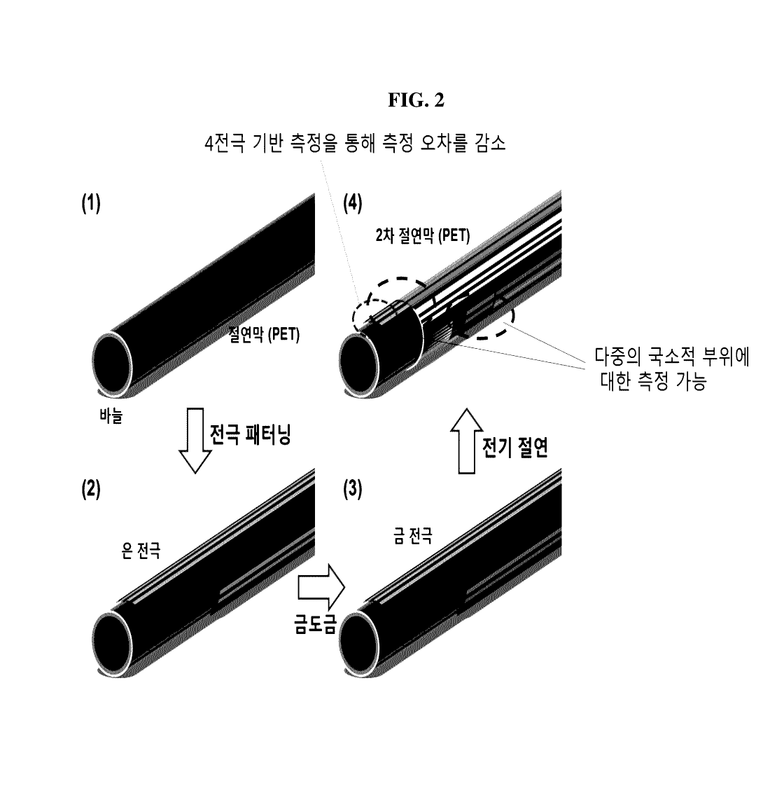

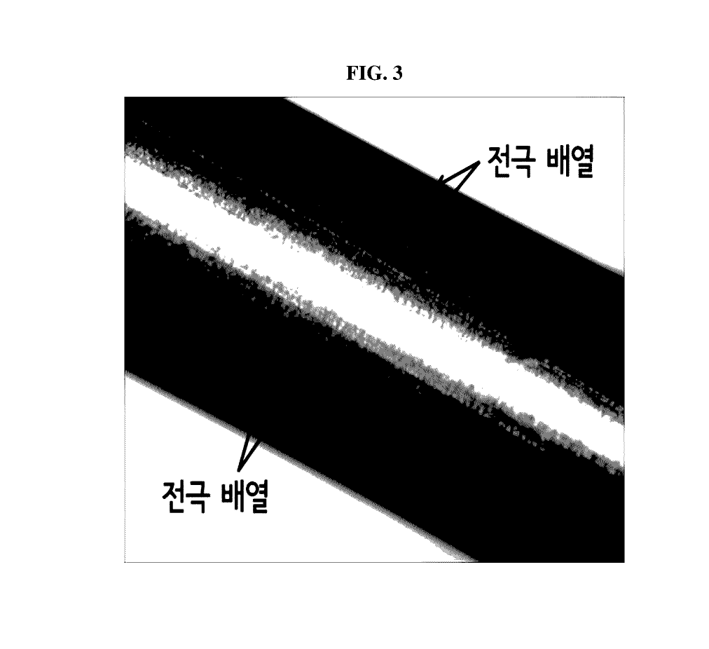

FIG. 1 is a schematic diagram of a biopsy needle according to an exemplary embodiment of the present invention, FIG. 2 is a manufacturing process diagram for the biopsy needle of FIG. 1, and FIG. 3 is a photograph illustrating an actual product for the biopsy needle of FIG. 1.

FIG. 1 illustrates a state in which a plurality of metal electrodes based on screen printing is formed on a biopsy needle made of stainless steel.

Hereinafter, a manufacturing process of the biopsy needle to which an impedance sensor is integrated will be described with reference to FIG. 2.

First, referring to FIG. 2(1), the needle uses a stainless steel (SUS304) needle with a diameter of 1.5 mm. A material of an electrical metal electrode may be variously used, but uses gold, stainless steel, or the like in which biocompatibility is guaranteed by considering that the biopsy needle is inserted into the body. For electrical insulation from the electrode to be formed below, a polyethylene terephthalate (PET) layer is coated by a thermal contraction method, and as illustrated in FIG. 2(2), an electrode pattern having a diameter of 100 μm and a length of 6 to 7 cm is patterned on the PET layer with silver paste by a screen printing method. Thereafter, the electrode pattern is sintered for 20 minutes at 120° C.

Here, the patterned silver electrode may be replaced with a general metal wire or a metal electrode.

As described above, in the present invention, screen printing or a silver/gold electrode and other wires such as SUS may be applied.

As an example of the electrode pattern, the electrode pattern is patterned at both an upper surface and a lower surface of the needle and tissues at different positions may be measured in real time by varying a length of the electrode pattern by about 1 cm.

Thereafter, as illustrated in FIG. 2(3), gold plating is performed on the silver electrode pattern, and then as illustrated in FIG. 2(4), the surface of the needle except for the electrode pattern portion for measuring is electrically insulated from the PET layer again. Here, in the case of silver, the silver pattern phase is covered with gold through gold plating to be suitable for the body because the biocompatibility is not yet guaranteed in an invasive medical device. In detail, the needle with the electrode pattern may be plated in a gold plating solution for 1,000 seconds at a current density of 1 mA/cm².

Referring again, in the present invention, the electrode array is formed through a process of printing metal paste for an electrode such as gold, silver, and the like or attaching a general metal wire and a metal electrode. In order to prevent a short-circuit with the biopsy needle, the surface of the biopsy needle is insulated by using a thermal contractive insulating tube such as the PET layer.

The electrode array formed on the needle is connected to an impedance analyzer to measure impedance of the tissue around the electrode. Further, since the electrode array may be formed to cover the peripheral portion of the biopsy needle, the impedance of the local biopsy tissue adjacent to the needle may be selectively measured. The measurement of the impedance may be implemented by selectively connecting the electrode array on the needle to the impedance analyzer by using a multiplexer and the like.

Referring to FIG. 3, the electrodes are formed by using screen printing and electrode paste on the biopsy needle having a diameter of 1.5 mm. A line width of each electrode is within 100 μm. It can be seen that the electrodes are printed on the biopsy needle through a photograph. As the electrode material, silver is used and gold, titanium, and the like which are biocompatible metal materials may be used. Further, it can be seen that the electrodes may be formed in several directions instead of one direction by repeating screen printing.

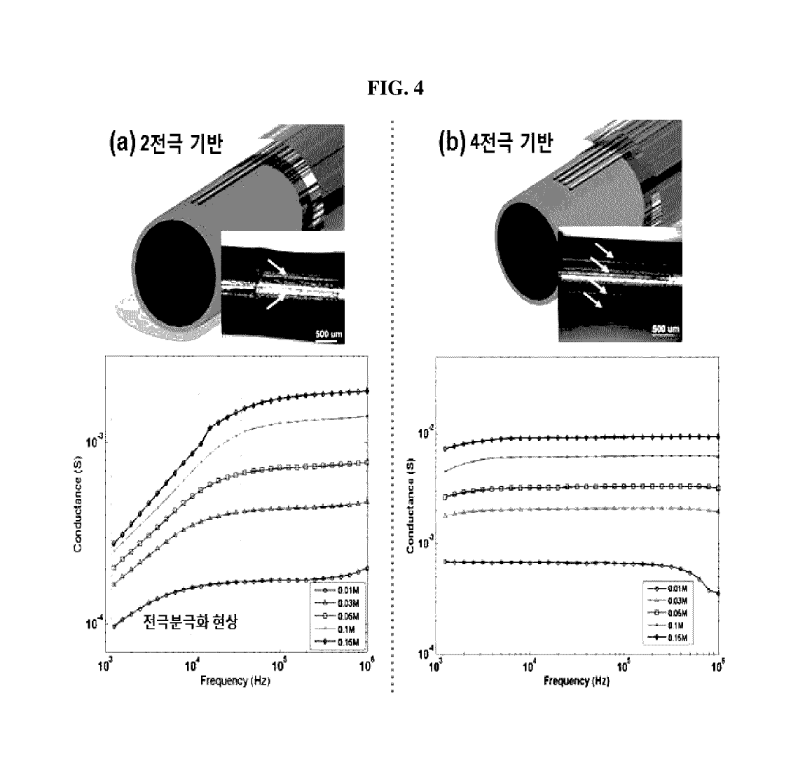

FIGS. 4A and 4B are diagrams illustrating an example of a biopsy needle for measuring impedance based on two electrodes and four electrodes and a result of measuring conductance of salt water. Hereinafter, measuring by a two-electrode method in which only two electrodes are formed on the surface of the needle and measuring by a four-electrode method in which four electrodes are formed on the surface of the needle are compared with each other.

In this measurement, conductance of salt water is used, and the reason is that there is no relaxation and the conductance is constant in a frequency range within 1 MHz. Accordingly, when salt water is measured by the biopsy needle with the electrode array, the measurement conductance needs to be constant in the frequency within 1 MHz.

In FIG. 4A, in the measuring by a two-electrode method in which only two electrodes are formed on the surface of the needle, it can be seen that an electrode polarization phenomenon is shown. The electrode polarization phenomenon is significant toward a low frequency to reduce the measured conductance. In detail, in the measuring result of the salt water, in the case of the two-electrode based measurement, it can be seen that the measured conductance is significantly reduced as the measured frequency is decreased.

Meanwhile, in the case of four-electrode based measuring using four electrodes formed as illustrated in FIG. 4B, it can be seen that the electrode polarization phenomenon is significantly reduced. As a result, it can be seen that the electrode polarization phenomenon having the two-electrode based measurement and the measurement error may be removed.

In the case of an actual saline, the concentration of sodium chloride (NaCl) is close to 0.15 M, and when the two-electrode measurement is performed in a salt solution of NaCl of 0.15 M, it can be seen that the electrode polarization phenomenon seriously occurs.

Accordingly, when measuring the vivo tissue based on two electrodes, a serious measurement error may occur, and in order to prevent the serious measurement error, it can be seen that the measurement error may be solved by measuring based on four electrodes by forming a plurality of electrodes.

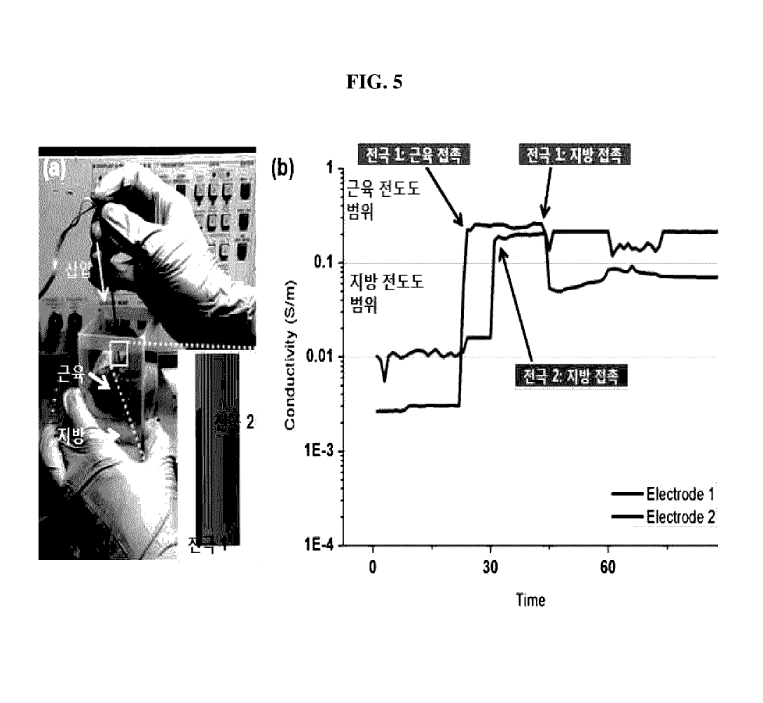

FIGS. 5A and 5B are diagrams illustrating an experiment for measuring impedance of a local tissue using a biopsy needle with a multiple electrode arrays and an experimental result.

Impedance of a pork tissue is measured in real time while the needle is pierced by using the needle manufactured in FIG. 5A. A plurality of electrode arrays is disposed along the top and bottom of the needle, and electrode 1 reaches an end of the needle and electrode 2 is disposed with a smaller length by about 1 cm. That is, two pairs of four electrode arrays are formed before and behind the needle at an interval of 1 cm. While the needle is pierced into a pork with muscles and fat, a change in impedance measured in each electrode is measured.

As a pork sample used in the present experiment, a part where the muscles and the fat are clearly divided is used, and a measuring frequency is 1 kHz. Generally, there is a difference according to the animal when the impedance is measured at 1 kHz, but generally, it is known that the muscles have specific conductance of 10⁻¹ to 10⁰ (S/m) and the fat has specific conductance of 10⁻³ to 10⁻¹ (S/m) (C. Gabriel et al., 1996, Phys. Med. Biol, 41, 2231-2249). Gabriel et al., 1996, Phys. Med. Biol, 41, 2231-2249).

FIG. 5B illustrates specific conductance of a meat tissue measured through each electrode array according to a depth inserted into the meat tissue. The measured conductance value is converted into the specific conductance by a cell constant measured through salt water.

When electrode 1 and electrode 2 contact the muscles, the specific conductance of 0.2 (S/m) is measured, and when only electrode 1 reaches the fat while the needle is further pierced, the specific conductance of 0.07 (S/m) is measured. In detail, when the needle is pierced into the meat, electrode 1 which is a longer electrode contacts the muscles, and as a result, the conductance corresponding to the muscle is measured, and thereafter, when the needle is further pierced, the conductance corresponding to the muscle is measured even in electrode 2 which is a shot electrode.

When the needle is further pierced, the conductance measured when electrode 1 contacts the fat part is rapidly reduced, and on the contrary, electrode 2 is still measured as a relatively high conductance value of the muscle part.

As a result, it can be seen that the impedance sensor integrated on the needle operates well and the impedance of the tissues at various positions may be measured in real time by several pairs of electrode patterns.

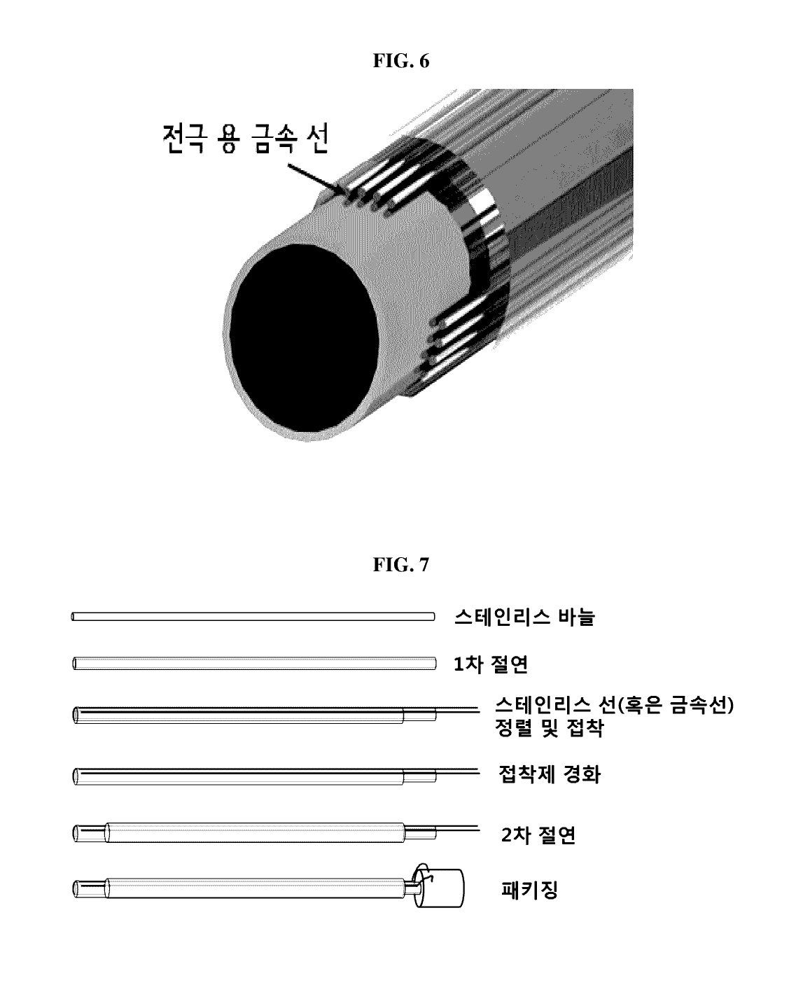

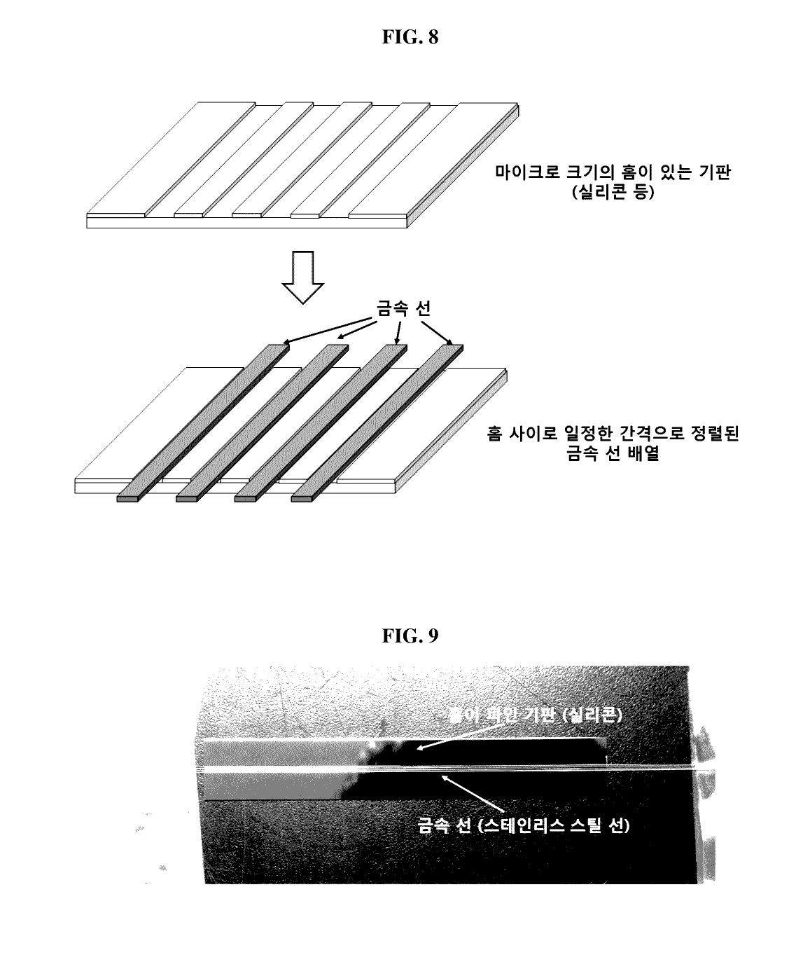

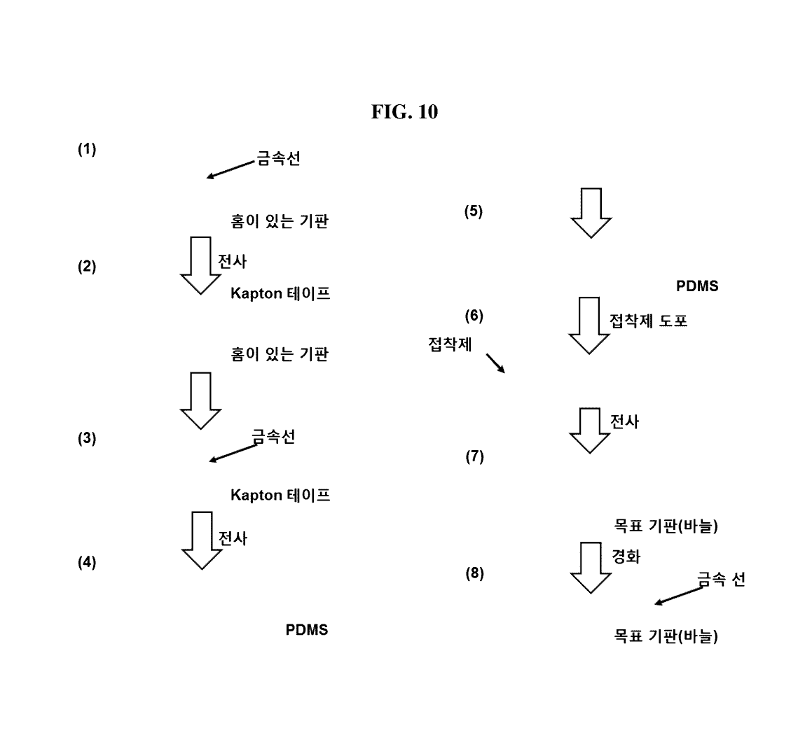

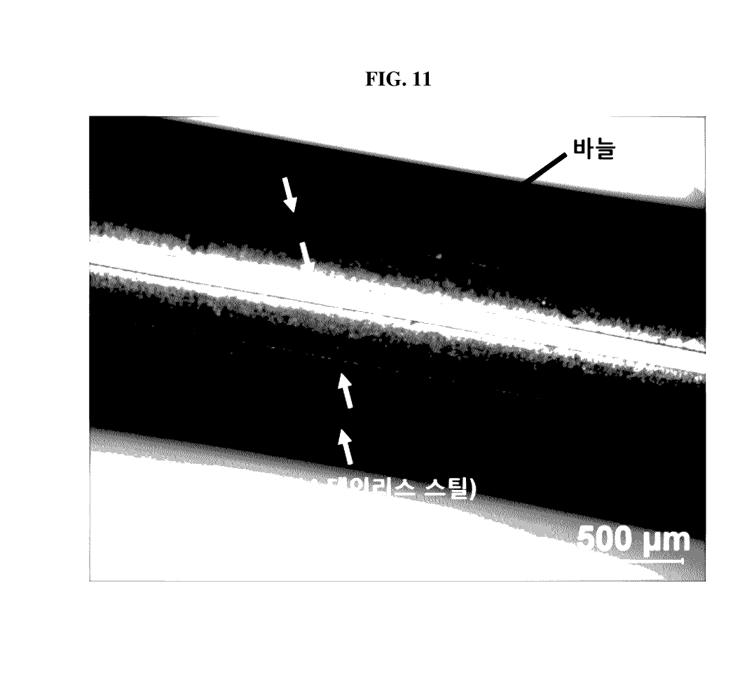

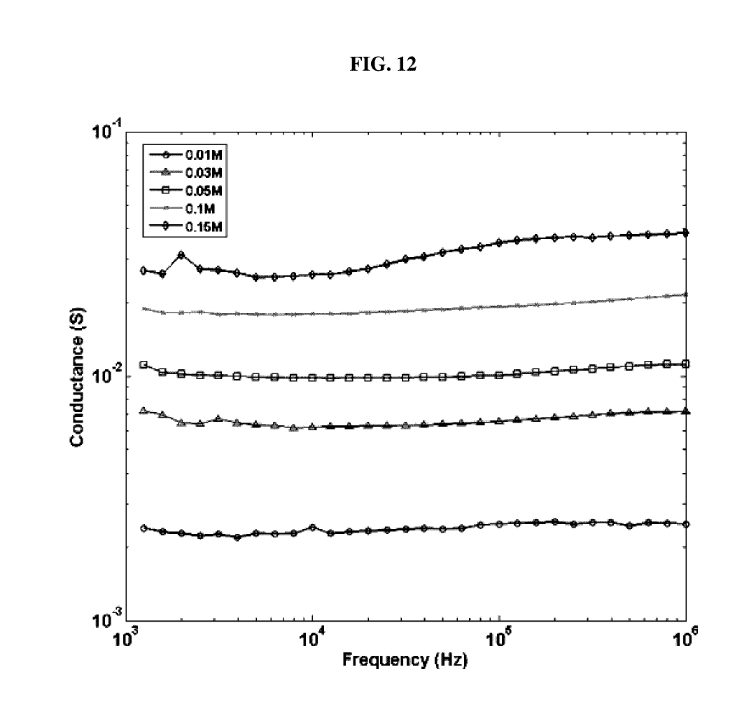

FIG. 6 is a schematic diagram of a biopsy needle according to another exemplary embodiment of the present invention, FIG. 7 is a manufacturing process diagram of a needle using a stainless steel wire or a metal wire as an electrode material, FIG. 8 is a schematic diagram for a method of arranging metal wires, FIG. 9 is a photograph illustrating an actual example of arrangement of metal wires, FIG. 10 is a process diagram in which arranged metal wires are attached onto the needle, FIG. 11 is a photograph illustrating an actual product for the needle to which the metal wires are attached by using a transfer process and an adhesive, and FIG. 12 is a graph illustrating a result of measuring conductance of salt water by using the needle to which the metal wire electrode is attached.

In the exemplary embodiment, a metal wire made of stainless steel, gold, and other metal materials is attached onto the surface of the biopsy needle by using an adhesive to form the electrode array. The metal wire applied to the exemplary embodiment may have a diameter within 110 μm. In the case of using the metal wire, the biopsy needle for sensing impedance may be manufactured at low prices as compared with a metal paste printing method, and in the case of stainless steel, there is an advantage in that a needle with reliability of which biocompatibility is known and the strength is high may be manufactured.

Hereinafter, a manufacturing process of the biopsy needle using a stainless steel wire or a metal wire as the electrode material will be described with reference to FIG. 7.

First, a stainless steel (SUS304) needle is prepared. A material of an electrical metal electrode may be variously used, but uses gold, stainless steel, or the like in which biocompatibility is guaranteed by considering that the biopsy needle is inserted into the body. For electrical insulation from the electrode to be formed below, primary insulation is performed by coating a polyethylene terephthalate (PET) layer by a thermal contraction method.

Thereafter, the arranged metal wires (stainless steel, gold, other metal material, and the like) are attached onto the surface of the needle by an adhesive. Next, the adhesive is cured, and the needle with the metal wire is manufactured through insulating and packaging processes. The adhesive may be a UV curable adhesive (Loctite 3321).

Referring to FIG. 8, since the metal wire has a small size within 100 μm, an arranging process is required before the attaching process. After the substrate which has a groove to fit the size of the metal wire is manufactured, an arranging method on the groove through the metal wire is used. An actual photograph to be arranged may be verified in FIG. 9.

Referring to FIG. 10, an attaching process on the needle by using the arranged metal wires is as follows.

First, the arranged metal wires are attached to a Kapton tape having adhesion to be removed from the substrate with the groove. As a result, the metal wires are fixed to the Kapton tape in the arranged state. Thereafter, when the Kapton tape is attached to a cured PDMS elastomer and then removed, the arranged metal wires remain on the PDMS as it is because adhesion between polydimethylsiloxane (PDMS) and the metal wire is stronger.

Thereafter, after the adhesive is coated on the PDMS and the metal wire, the metal wires remain on the needle which is the target substrate while being attached and cured on the needle which is the target substrate.

The PDMS used in the present invention is cured during UV curing due to UV transmissivity, and when the PDMS does not adhere to the used adhesive to be removed after curing, only the metal wires are attached to the needle. A photograph of an actual needle product manufactured by using the process is illustrated in FIG. 11.

Referring to FIG. 12, meanwhile, as the result for the electrodes using the metal electrodes based on screen printing, similarly to FIG. 4B, it can be seen that the electrode polarization phenomenon is significantly reduced.

Further, the actual tissue may be measured by using the biopsy needle according to the present invention, and several local parts of the tissue around the needle which is impossible existing studies may be selectively measured. Further, in the present invention, the electrode polarization error phenomenon which has become a problem under the existing two-electrode measurement is removed by the four-electrode measurement.

As described above, it is possible to selectively measure a predetermined tissue around the needle according to a direction of electrode formation and the number of electrodes while measuring impedance by using a plurality of electrodes by proposing a biopsy needle with a plurality of electrode arrays on a surface.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims.

The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. A biopsy needle with an electrode array which measures impedance for a plurality of biopsy points in real time, wherein
the needle includes electrode patterns configured by a plurality of electrode arrays on the surface, and
the electrode patterns are spaced apart from each other along the top and bottom of the needle.

2. The biopsy needle of claim 1, wherein the plurality of electrode arrays is patterned at different positions on the needle.

3. The biopsy needle of claim 1, wherein the electrode pattern is a metal paste or a metal wire for the electrode including any one of gold, silver, and stainless steel.

4. The biopsy needle of claim 3, wherein the metal wires include any one of stainless steel, gold, silver, and other metals including different metal materials, and the metal wires are attached onto the needle by using an adhesive.

5. The biopsy needle of claim 3, wherein a material of the needle includes stainless steel.

6. The biopsy needle of claim 1, wherein the electrode pattern is a four-electrode method with four electrodes.

7. The biopsy needle of claim 1, wherein an electrical insulating layer is formed on the surface of the needle except for the electrode pattern portions.

8. A method of manufacturing a biopsy needle with an electrode array, which measures impedance for a plurality of biopsy points in real time, the method comprising:
preparing a needle body; and
patterning electrode patterns configured by a plurality of electrode arrays on the needle body,
wherein the electrode patterns are spaced apart from each other along the top and bottom of the needle.

9. The method of claim 8, wherein the patterning of the electrode patterns includes patterning a first electrode pattern on the needle body with a metal paste and depositing a second electrode pattern on the first electrode pattern by a plating method.

10. The method of claim 9, wherein the first electrode pattern is a silver paste, and the second electrode pattern is a gold-plated pattern.

11. The method of claim 9, further comprising:
forming an electrical insulating layer on the surface of the needle body except for the electrode pattern portions before and after the patterning of the electrode patterns.

12. The method of claim 9, wherein the electrode pattern is a four-electrode type with four electrodes.

13. The method of claim 8, wherein the patterning of the electrode patterns includes attaching the arranged metal wires on the needle body by using an adhesive, curing the adhesive, and forming an electrical insulating layer on the surface of the needle body except for the arranged metal wire portion.

14. The method of measuring impedance of a biopsy tissue using the biopsy needle of claim 1, the method comprising:
measuring a change in impedance which is measured in each electrode array through a process of sequentially inserting a plurality of electrode arrays forming the electrode patterns into the biopsy tissue.

15. The method of measuring impedance of a biopsy tissue using the biopsy needle of claim 2, the method comprising:
measuring a change in impedance which is measured in each electrode array through a process of sequentially inserting a plurality of electrode arrays forming the electrode patterns into the biopsy tissue.

16. The method of measuring impedance of a biopsy tissue using the biopsy needle of claim 3, the method comprising:
measuring a change in impedance which is measured in each electrode array through a process of sequentially inserting a plurality of electrode arrays forming the electrode patterns into the biopsy tissue.

17. The method of measuring impedance of a biopsy tissue using the biopsy needle of claim 4, the method comprising:
measuring a change in impedance which is measured in each electrode array through a process of sequentially inserting a plurality of electrode arrays forming the electrode patterns into the biopsy tissue.

18. The method of measuring impedance of a biopsy tissue using the biopsy needle of claim 5, the method comprising:
measuring a change in impedance which is measured in each electrode array through a process of sequentially inserting a plurality of electrode arrays forming the electrode patterns into the biopsy tissue.

19. The method of measuring impedance of a biopsy tissue using the biopsy needle of claim 6, the method comprising:
measuring a change in impedance which is measured in each electrode array through a process of sequentially inserting a plurality of electrode arrays forming the electrode patterns into the biopsy tissue.

20. The method of measuring impedance of a biopsy tissue using the biopsy needle of claim 7, the method comprising:
measuring a change in impedance which is measured in each electrode array through a process of sequentially inserting a plurality of electrode arrays forming the electrode patterns into the biopsy tissue.
